(19) United States
(12) Patent Application Publication
(10) Pub. No.: US 2024/0408123 A1
(43) Pub. Date: Dec. 12, 2024

(54) NUCLEOTIDES AND OLIGOSACCHARIDES FOR USE AS FOOD COMPOSITION

(71) Applicant: MARS, INCORPORATED, McLean, VA (US)

(72) Inventor: Willy Joly, Aimargues (FR)

(73) Assignee: MARS, INCORPORATED, McLean, VA (US)

(21) Appl. No.: 18/700,603

(22) PCT Filed: Oct. 18, 2022
(86) PCT No.: PCT/US2022/046986

§ 371 Date: Apr. 11, 2024

(30) Foreign Application Priority Data
Oct. 22, 2021 (EP) 21204186.7

(51) Int. Cl. A61K 31/702; A23K 20/132; A23K 20/142; A23K 20/163; A23K 20/174; A23K 20/179; A23K 50/42; A61K 36/064; A61K 39/39; A61P 37/04

(52) U.S. Cl. CPC A61K 31/702; A23K 20/132; A23K 20/142; A23K 20/163; A23K 20/174; A23K 20/179; A23K 50/42; A61K 36/064; A61K 39/39; A61P 37/04

(57) ABSTRACT

The present invention relates to a food composition comprising, in % by weight of the food composition on dry matter basis, i) nucleotides in an amount of at least about 0.05% and ii) oligosaccharides in an amount of at least about 0.1%, wherein said oligosaccharides are selected from xylo-oligosaccharides (XOS), fructo-oligosaccharides (FOS), galacto-oligosaccharides (GOS), and combinations thereof.

### CROSS-REFERENCE TO RELATED APPLICATIONS

This application claims the benefit of priority to EP Patent Application Serial No. 21204186.7, filed on Oct. 22, 2021, the contents of which are incorporated herein by reference in their entirety.

### FIELD OF THE INVENTION

Various embodiments of the disclosure relate to the field of food products and compositions, such as food additives and supplements, and uses thereof. Those food compositions are particularly convenient for animals, especially companion animals. The present disclosure further relates to food products and compositions which are beneficial for health. More particularly, the present disclosure relates to food products and compositions for improving the immune system and vaccine response, especially at an early age.

### BACKGROUND OF THE INVENTION

The immune system's main role is preventing and fighting diseases and is a good indicator of overall health. It is now established that nutrition has the capacity to modulate immune function in animals.

For example, resistance to a challenge has been observed with Candida albicans in Fanslow et al. (“Effect of Nucleotide Restriction and Supplementation on Resistance to Experimental Murine Candidiasis”; Journal of Parenteral and Enteral Nutrition; Vol. 12, No. 1; 1988), and Staphylococcus aureus in Kulkarni et al. (“Effect of Dietary Nucleotides on Response to Bacterial Infections”; Journal of Parenteral and Enteral Nutrition; Vol. 10, No. 2; 1986) by feeding different nucleotide enriched diets to mice.

Dietary nucleotides (e.g. purines, pyrimidines and mixtures thereof) have further been shown to be metabolized in rats in most body tissues, including glandular and lymphoid tissues, in Savaiano et al. (“Metabolism of orally and intravenously administered purines in rats”. The Journal of Nutrition. 110, 1793-804; 1980).

A more recent study on beagle puppies has shown that dietary supplementation with nucleotides mimicking the nucleotide composition of female dog's milk, improves the immune response capacity of puppies; as reported in Romano et al. (“Dietary nucleotides improve the immune status of puppies at weaning”; Journal of Animal Physiology and Animal Nutrition; Vol. 91, Issue 3-4, pages 158-162; 2007).

A study on cats has also shown that a supplementation diet including nucleotides leads to an increase in T lymphocytes proliferation together with an increase in leucocyte phagocytosis; as reported in Rutherfurd-Markwicka et al. (“The potential for enhancement of immunity in cats by dietary supplementation”; Veterinary Immunology and Immunopathology; 152, 333-340; 2013).

During the weaning period, while the food shifts from milk to solid, gastrointestinal disturbances can also increase the sensitivity to diarrhea and infection. Accordingly, the effect of some carbohydrates & oligosaccharides on the immune function has further been reported.

For example, a long-lasting effect of perinatal short-chain fructo-oligosaccharides (scFOS) supplementation on microbiota metabolite production, more than two months after the end of supplementation, is reported in Le Bourgot et al. (“Maternal short-chain fructooligosaccharide supplementation influences intestinal immune system maturation in piglets”; PLoS One 9, 2014 Sep. 19; 9(9):e107508. doi: 10.1371/journal.pone.0107508. eCollection 2014).

In pigs, maternal short-chain fructo-oligosaccharides (scFOS) supplementation is reported to induce an increased metabolic activity of the offspring microbiota during lactation with a higher butyrate production after weaning, compared to offspring of non-supplemented sows; as reported in Le Bourgot et al. (“Maternal short-chain fructooligosaccharide supplementation increases intestinal cytokine secretion, goblet cell number, butyrate concentration and Lawsonia intracellularis humoral vaccine response in weaned pigs”. British Journal of Nutrition; 117, 83-92; 2017).

Moreover, post-weaning scFOS diet has been reported to increase anti-influenza IgA levels in pig serum and faeces; as reported in Le Bourgot et al. (“Short-chain fructooligosaccharide supplementation during gestation and lactation or after weaning differentially impacts pig growth and IgA response to influenza vaccination”. Journal of Functional Foods; 24, 307-315; 2016). Still, the study appears to conclude that, unlike direct scFOS supplementation of the weaning diet, scFOS supplementation of the maternal diet did not modify the response of offspring to influenza vaccine and was not synergistic with post-weaning scFOS supplementation.

Also, Ebersbach et al. (“Certain dietary carbohydrates promote Listeria infection in a guinea pig model, while others prevent it”; International Journal of Food Microbiology; 140, 218-224; 2010) reports that the addition of dietary xylo-oligosaccharides (XOS) and galacto-oligosaccharides (GOS) significantly increases resistance to Listeria monocytogenes; although the effect was not reproduced with other carbohydrates such as inulin, apple pectin and/or polydextrose.

Overall, the challenges of food supplementation to improve the immune system in animals remain important because very little data is available on the development of the immune system in puppies and kittens or on the development and establishment of the intestinal flora and its impact on the digestive and immune systems during growth. Furthermore, these data are complex to acquire as a sufficient number of animals must be available, taking into account significant inter-individual variability on these parameters.

Furthermore, it is important to underline the difficulty of promoting an improvement of the immune system in order to improve the vaccine response through nutrition in growing animals. Indeed, in young animals, both innate and adaptive immune systems are being developed. The maturity of these systems is acquired after different stages during which an immature cell will eventually acquire a specific cell type. It is therefore quite complex to influence the development of these systems.

There is thus a need for novel active ingredients or combinations thereof, which can have an effect in modulating the immune function in animals; especially the immune function at an early stage, and for a broad selection of animals, such as companion animals.

Also, there is a need for novel active ingredients or combinations thereof, for which the expected effect on health can be applicable to a plurality of companion animals, especially dogs, cats, and the like.

There is also a need for novel active ingredients or combinations thereof, which can improve the vaccine response of an animal toward, especially toward a plurality of antigens or pathogens.

There is also a need for compositions which can be administered as food compositions and food supplements to animals; more particularly compositions which possess sufficient palatability.

There is also a need for compositions which are manufactured from, or derived from, natural sources.

The disclosure has for purpose to meet the above-mentioned needs.

### SUMMARY OF THE DISCLOSURE

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

A first aspect of the present disclosure relates to a food composition including, in % by weight of the food composition on dry matter basis, i) nucleotides in an amount of at least about 0.05% and ii) oligosaccharides in an amount of at least about 0.1%, wherein said oligosaccharides can be selected from xylo-oligosaccharides (XOS), fructo-oligosaccharides (FOS), galacto-oligosaccharides (GOS), and combinations thereof.

Another aspect of the present disclosure relates to the said food composition, including, in % by weight of the food composition on dry matter basis, i) nucleotides in an amount of at least about 0.05% and ii) oligosaccharides in an amount of at least about 0.1%, wherein said oligosaccharides can be selected from xylo-oligosaccharides (XOS), fructo-oligosaccharides (FOS), galacto-oligosaccharides (GOS), and combinations thereof; for use as a medicament; in particular a medicament for improving the immune function of a companion animal.

Another aspect of the present disclosure relates to a use of the said food composition, including, in % by weight of the food composition on dry matter basis, i) nucleotides in an amount of at least about 0.05% and ii) oligosaccharides in an amount of at least about 0.1%, wherein said oligosaccharides can be selected from xylo-oligosaccharides (XOS), fructo-oligosaccharides (FOS), galacto-oligosaccharides (GOS), and combinations thereof; for the preparation of a medicament; in particular for improving the immune function of a companion animal.

Another aspect of the present disclosure relates to a kit for manufacturing a food composition, including:
(i) a source of nucleotides;
(ii) a source of oligosaccharides selected from xylo-oligosaccharides (XOS), fructo-oligosaccharides (FOS), galacto-oligosaccharides (GOS), and combinations thereof.

Another aspect of the present disclosure relates to a method of manufacturing the said food composition, including:
a) mixing
a source nucleotides;
a source of oligosaccharides selected from xylo-oligosaccharides (XOS), fructo-oligosaccharides (FOS), galacto-oligosaccharides (GOS), and combinations thereof;
b) heating the mixture, thereby manufacturing the food composition.

Another aspect of the present disclosure relates to a method for improving the immune function of a companion animal, the method including: (a) providing a food composition including at least, in % by weight of the food composition on dry matter basis, i) nucleotides in an amount of at least about 0.05% and ii) oligosaccharides in an amount of at least about 0.1%, wherein said oligosaccharides are selected from xylo-oligosaccharides (XOS), fructo-oligosaccharides (FOS), galacto-oligosaccharides (GOS), and combinations thereof, and (b) administering to the companion animal an effective amount of the said food composition.

It is to be understood that both the foregoing general description and the following detailed description are exemplary and explanatory only and are not restrictive of the disclosed embodiments, as claimed.

### DESCRIPTION OF THE FIGURES

FIG. 1 is a graphical depiction of the Vaccination protocol for kittens (European cats).

FIG. 2 is a graphical depiction of the assessment of the chlamydia antibodies titre by ELISA following vaccine administration in a group of kittens.

FIG. 3 is a graphical depiction of cytokine responses, as measured with the Luminex assay ex vivo on blood samples performed in 3 conditions: not stimulated; stimulated with PHA; and stimulated with rabies antigen (RA).

### DETAILED DESCRIPTION OF THE DISCLOSURE

Although some oligosaccharides such as xylo-oligosaccharides (XOS) and short-chain fructo-oligosaccharides (scFOS), and nucleotides, are thought to have a positive effect on health, separately and independently on the immune system and on the gut health, their combinatorial use has not been studied yet in animals.

The present disclosure describes in vivo and ex vivo clinical studies reporting the combinatory effect of a nucleotide source, and of an oligosaccharide source, said oligosaccharide being selected from xylo-oligosaccharides (XOS), fructo-oligosaccharides (FOS), galacto-oligosaccharides (GOS), and combinations thereof.

This proof of efficacy demonstrates the potential for a combination of (i) nucleotides and (ii) oligosaccharides selected from xylo-oligosaccharides (XOS), fructo-oligosaccharides (FOS), galacto-oligosaccharides (GOS) to impact positively immune function directly, especially in the context of vaccine administration and modulation of the vaccine response. Advantageously, this scientific evidence demonstrates a positive effect in a plurality of companion animals, including cats (kitten).

In particular, it is reported herein the effect of this combination of active ingredients in companion animals, especially companion animals at an early stage of development, for improving the immune function.

According to an aspect, the food composition according to the present disclosure can include, in % by weight of the food composition on dry matter basis, i) nucleotides in an amount of at least about 0.05% and ii) oligosaccharides in an amount of at least about 0.1%, wherein said oligosaccharides can be selected from xylo-oligosaccharides (XOS), fructo-oligosaccharides (FOS), galacto-oligosaccharides (GOS), and combinations thereof.

According to some embodiments, the food composition according to the present disclosure can include, in % by weight of the food composition on dry matter basis, i) nucleotides in an amount of at least about 0.06%, 0.07%, 0.08%, 0.09%, 0.1%, 0.2%, 0.3%, 0.4%, or 0.5% and ii) oligosaccharides in an amount of at least about 0.1%, wherein said oligosaccharides can be selected from xylo-oligosaccharides (XOS), fructo-oligosaccharides (FOS), galacto-oligosaccharides (GOS), and combinations thereof.

According to some embodiments, the food composition according to the present disclosure can include, in % by weight of the food composition on dry matter basis, i) nucleotides in an amount of at least about 0.05% and ii) oligosaccharides in an amount of at least about 0.2%, 0.3%, 0.4%, 0.5%, 0.6%, 0.7%, 0.8%, 0.9%, wherein said oligosaccharides can be selected from xylo-oligosaccharides (XOS), fructo-oligosaccharides (FOS), galacto-oligosaccharides (GOS), and combinations thereof.

In certain embodiment, the recited compositions can be dry compositions.

According to some embodiments, the food composition according to the present disclosure can include, in % by weight of the food composition on dry matter basis, i) nucleotides in an amount of at least about 0.05% and ii) oligosaccharides in an amount of at least about 0.1%, wherein said oligosaccharides can be selected from xylo-oligosaccharides (XOS), fructo-oligosaccharides (FOS), and combinations thereof.

According to some embodiments, the food composition according to the present disclosure can include, in % by weight of the food composition on dry matter basis, i) nucleotides in an amount of at least about 0.05% and ii) fructo-oligosaccharides (FOS) in an amount of at least about 0.1%.

According to some embodiments, the food composition according to the present disclosure can include, in % by weight of the food composition on dry matter basis, i) nucleotides in an amount of at least about 0.05% and ii) xylo-oligosaccharides (XOS) in an amount of at least about 0.1%.

According to some embodiments, the food composition according to the present disclosure can include, in % by weight of the food composition on dry matter basis, i) nucleotides in an amount of at least about 0.05% and ii) galacto-oligosaccharides (GOS) in an amount of at least about 0.1%.

In certain embodiments, the food composition according to the present disclosure can include, in % by weight of the food composition on dry matter basis, i) nucleotides in an amount ranging from about 0.05% to about 0.5%, and ii) oligosaccharides in an amount ranging from about 0.1% to about 2%, wherein said oligosaccharides can be selected from xylo-oligosaccharides (XOS), fructo-oligosaccharides (FOS), galacto-oligosaccharides (GOS), and combinations thereof.

In certain embodiments, the food composition according to the present disclosure can include, in % by weight of the food composition on dry matter basis, i) nucleotides in an amount ranging from about 0.05% to about 0.5%, and ii) oligosaccharides in an amount ranging from about 0.1% to about 2%, wherein said oligosaccharides can be selected from xylo-oligosaccharides (XOS), fructo-oligosaccharides (FOS), and combinations thereof.

For example, the food composition according to the present disclosure can include, in % by weight of the food composition on dry matter basis, i) nucleotides in an amount ranging from about 0.05% to about 0.5%, and ii) fructo-oligosaccharides (FOS) in an amount ranging from about 0.1% to about 2%.

For example, the food composition according to the present disclosure can include, in % by weight of the food composition on dry matter basis, i) nucleotides in an amount ranging from about 0.05% to about 0.5%, and ii) xylo-oligosaccharides (XOS) in an amount ranging from about 0.1% to about 2%.

In certain embodiments, the food composition according to the present disclosure, can include, in % by weight of the food composition on dry matter basis:
(i) nucleotides in an amount of at least about 0.05%;
(ii) xylo-oligosaccharides (XOS) in an amount of at least about 0.1%, and/or
(iii) fructo-oligosaccharides (FOS) in an amount of at least about 0.3%.

According to some embodiments, the food composition according to the present disclosure, can include, in % by weight of the food composition on dry matter basis:
(i) nucleotides in an amount of at least about 0.05%;
(ii) xylo-oligosaccharides (XOS) in an amount of at least about 0.1%, and
(iii) fructo-oligosaccharides (FOS) in an amount of at least about 0.3%.

According to some embodiments, the food composition according to the present disclosure, can include, in % by weight of the food composition on dry matter basis:
(i) nucleotides in an amount of at least about 0.05%;
(ii) fructo-oligosaccharides (FOS) in an amount of at least about 0.3%.

In certain embodiments, the food composition according to the present disclosure, can include, in % by weight of the food composition on dry matter basis:
(i) nucleotides in an amount ranging from about 0.05% to about 0.5%;
(ii) xylo-oligosaccharides (XOS) in an amount ranging from about 0.1% to about 0.7%, and/or
(iii) fructo-oligosaccharides (FOS) in an amount ranging from about 0.3% to about 1%.

According to some embodiments, the food composition according to the present disclosure, can include, in % by weight of the food composition on dry matter basis:
(i) nucleotides in an amount ranging from about 0.05% to about 0.5%;
(ii) xylo-oligosaccharides (XOS) in an amount ranging from about 0.1% to about 0.7%.

According to some embodiments, the food composition according to the present disclosure, can include, in % by weight of the food composition on dry matter basis:
(i) nucleotides in an amount ranging from about 0.05% to about 0.5%;
(ii) fructo-oligosaccharides (FOS) in an amount ranging from about 0.3% to about 1%.

In certain embodiments, the present disclosure relates to a food composition, wherein the nucleotides can be from a source of nucleotides from a yeast extract or a fraction thereof, in particular a yeast extract selected from a Kluyveromyces yeast extract and/or a Saccharomyces cerevisae yeast extract, or a fraction thereof.

In various embodiments, the present disclosure relates to a food composition, wherein said nucleotides can include a combination of pyrimidines and purines.

In various embodiments, the present disclosure relates to a food composition, wherein said nucleotides can include a combination of pyrimidines and purines with a weight ratio of pyrimidines to purines ranging from about 9:1 to about 1:1.

According to some embodiments, the nucleotides can include a combination of pyrimidines and purines with a weight ratio of pyrimidines to purines ranging from about 9:1 to about 1:1; which includes without limitation 9:1, 8:1, 7:1, 6:1, 5:1, 4:1, 3:1, 2:1, 1:1.

In certain embodiments, the present disclosure relates to a food composition, wherein said nucleotides can include pyrimidines selected from cytidine monophosphate (CMP), uridine monophosphate (UMP), thymidine monophosphate (TMP), and/or combinations thereof.

In certain embodiments, the present disclosure relates to a food composition, wherein said nucleotides can include purines selected from adenosine monophosphate (AMP), guanosine monophosphate (GMP), inosine monophosphate (IMP), xanthosine monophosphate (XMP), and/or combinations thereof.

In certain embodiments, the present disclosure relates to a food composition, wherein the oligosaccharides can be selected from xylo-oligosaccharides (XOS), fructo-oligosaccharides (FOS), and/or combinations thereof.

Short-chain oligosaccharides and long-chain oligosaccharides can be considered according to the present disclosure.

In certain embodiments, the present disclosure relates to a food composition, wherein said oligosaccharides can include a number of saccharide units of at least 2 units; for example ranging from 2 to 10, which thus can include 2, 3, 4, 5, 6, 7, 8, 9 or 10. Alternatively, said oligosaccharides can include a number of saccharide units of 10 or above 10.

In certain embodiments, the present disclosure relates to a food composition, wherein said XOS can include XOS composed of xylose units linked by β-(1,4) bonds, with a degree of polymerization (DP) ranging from 2 to 10; which thus can include 2, 3, 4, 5, 6, 7, 8, 9 and 10.

In certain embodiments, the present disclosure relates to a food composition, wherein said XOS can include 2 to 7 xylose molecules linked by β-1,4 glycosidic bonds.

In certain embodiments, the present disclosure relates to a food composition, wherein said FOS can include at least about 50% of short-chain fructo-oligosaccharides (scFOS) in % by weight of the FOS in the food composition on dry matter basis.

In certain embodiments, the present disclosure relates to a food composition, wherein said FOS can include at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85% 86%, 87%, 88%, 89% or even 90% of short-chain fructo-oligosaccharides (scFOS) in % by weight of the FOS in the food composition on dry matter basis

In certain embodiments, the present disclosure relates to a food composition, wherein said scFOS can include scFOS with 2, 3 or 4 fructose units linked to one glucose unit (also referred respectively as GF2, GF3 and/or GF4).

In certain embodiments, the food composition according to the present disclosure, can further include at least one ingredient selected from vitamine E, arginine and beta-carotene. More particularly, the food composition according to the present disclosure, can further include at least two ingredients selected from vitamin E, arginine and beta-carotene.

In certain embodiments, the food composition according to the present disclosure, can further include vitamin E in an amount ranging from about 0.01% to about 0.1%, in % by weight of the food composition on dry matter basis; which can include 0.01%, 0.02%, 0.03%, 0.04%, 0.05%, 0.06%, 0.07%, 0.08%, 0.09% and 0.1% in % by weight of the food composition on dry matter basis.

In certain embodiments, the food composition according to the present disclosure, can further include beta-carotene in an amount ranging from about 0.0001% to about 0.001%, in % by weight of the food composition on dry matter basis.

In certain embodiments, the food composition according to the present disclosure, can further include arginine in an amount ranging from about 0.1% to about 10%, in % by weight of the food composition on dry matter basis; which includes 0.1%, 0.2%, 0.3%, 0.4%, 0.5%, 0.6%, 0.7%, 0.8%, 0.9%, 1%, 2%, 3%, 4%, 5%, 6%, 7% 8%, 9% and 10% by weight of the food composition on dry matter basis.

In certain embodiments, the food composition according to the present disclosure, can further include:
vitamin E in an amount ranging from about 0.01% to about 0.1%, in % by weight of the food composition on dry matter basis; which can include 0.01%, 0.02%, 0.03%, 0.04%, 0.05%, 0.06%, 0.07%, 0.08%, 0.09% and 0.1% in % by weight of the food composition on dry matter basis; and/or
beta-carotene in an amount ranging from about 0.0001% to about 0.001%, in % by weight of the food composition on dry matter basis; and/or
arginine in an amount ranging from about 0.1% to about 10%, in % by weight of the food composition on dry matter basis; which includes 0.1%, 0.2%, 0.3%, 0.4%, 0.5%, 0.6%, 0.7%, 0.8%, 0.9%, 1%, 2%, 3%, 4%, 5%, 6%, 7%, 8%, 9% and 10% by weight of the food composition on dry matter basis.

In certain embodiments, the present disclosure relates to a food composition, wherein said food composition can be a dry composition, a wet composition, or a semi-moist composition; such as a dry composition.

In certain embodiments, the present disclosure relates to a food composition, wherein said food composition can be a nutritionally complete food or a functional complement.

In another aspect, the present disclosure relates to a food composition, for its use as a medicament.

In certain embodiments, the present disclosure relates to a food composition, for its use in a method for improving the immune function of a companion animal.

In certain embodiments, the present disclosure relates to a food composition, for its use in a method for improving the immune function, wherein the immune function can be selected from the innate immune function of the companion animal, the adaptive immune function of the companion animal, or both.

In certain embodiments, the present disclosure relates to a food composition, for its use in a method for improving the immune function, wherein the immune function can be selected from: cytokine production and antibody response. In an embodiment, the present disclosure relates to a food composition, for its use in a method for improving the antibody response to a vaccine.

In certain embodiments, the present disclosure relates to a food composition, for its use in a method for improving the immune response of a companion animal to an antigen. In an embodiment, the present disclosure relates to a food composition, for its use in a method for improving the immune response of a companion animal to an antigen from a pathogen or a vaccine directed against said pathogen.

In certain embodiments, the present disclosure relates to a food composition, for its use in a method for improving the immune response of a companion animal to an antigen from one or more pathogens and/or microorganisms and/or a vaccine directed against said pathogen(s) and/or microorganism(s), which can be selected from, without limitation: feline panleukopenia virus, herpesvirus, calicivirus and rabies virus or combination thereof.

According to exemplary embodiments, the disclosure relates to a food composition, for its use in a method for improving the immune response of a companion animal to an antigen from one or more bacteria selected from the genus Chlamydia; such as Chlamydophila felis.

In certain embodiments, the disclosure relates to a food composition, for its use in a method for preventing or treating immune deficiency in a companion animal.

In certain embodiments, the present disclosure relates to a food composition, wherein the companion animal can be selected from a canine or a feline, such as without limitation a cat or a dog.

According to exemplary embodiments, the companion animal can be a cat.

In certain embodiments, the disclosure relates to a food composition, wherein the companion animal can be a youngling.

In addition, according to another aspect, the present disclosure relates to a therapeutic use of a food composition as described, namely a food composition including, in % by weight of the food composition on dry matter basis, i) nucleotides in an amount of at least about 0.05% and ii) oligosaccharides in an amount of at least about 0.1%, wherein said oligosaccharides can be selected from xylo-oligosaccharides (XOS), fructo-oligosaccharides (FOS), galacto-oligosaccharides (GOS), and combinations thereof; as a medicament.

In certain embodiments, the present disclosure relates to a therapeutic use of a food composition as described; for improving the immune function of a companion animal.

In certain embodiments, the present disclosure relates to a therapeutic use of a food composition as described; for improving the immune response of a companion animal to an antigen

In certain embodiments, the present disclosure relates to a therapeutic use of a food composition as described; for preventing or treating immune deficiency in a companion animal.

Moreover, according to another aspect, the present disclosure relates to a therapeutic method including administering, to a companion animal, a food composition as described, namely a food composition including, in % by weight of the food composition on dry matter basis, i) nucleotides in an amount of at least about 0.05% and ii) oligosaccharides in an amount of at least about 0.1%, wherein said oligosaccharides can be selected from xylo-oligosaccharides (XOS), fructo-oligosaccharides (FOS), galacto-oligosaccharides (GOS), and combinations thereof, as a medicament.

In certain embodiments, the present disclosure relates to a therapeutic method for improving the immune function, including administering, to a companion animal, a food composition as described.

In certain embodiments, the present disclosure relates to a therapeutic method for improving the immune response to an antigen, including administering, to a companion animal, a food composition as described.

In certain embodiments, the present disclosure relates to a therapeutic method for preventing or treating immune deficiency, including administering, to a companion animal, a food composition as described.

In another aspect, the present disclosure provides a method for improving the immune function in a companion animal, the method including: (a) providing a food composition including at least, in % by weight of the food composition on dry matter basis, i) nucleotides in an amount of at least about 0.05% and ii) oligosaccharides in an amount of at least about 0.1%, wherein said oligosaccharides are selected from xylo-oligosaccharides (XOS), fructo-oligosaccharides (FOS), galacto-oligosaccharides (GOS), and combinations thereof, and (b) administering to the companion animal an effective amount of the said food composition.

In certain embodiments, the immune function to be improved can be selected from the innate immune function of the companion animal, the adaptive immune function of the companion animal, or both.

In some embodiments, the immune function to be improved can be selected from: cytokine production and antibody response.

In certain embodiments, the present disclosure provides a method for improving and/or increasing and/or eliciting the antibody response to a vaccine in a companion animal, the method including: (a) providing a food composition including at least, in % by weight of the food composition on dry matter basis, i) nucleotides in an amount of at least about 0.05% and ii) oligosaccharides in an amount of at least about 0.1%, wherein said oligosaccharides are selected from xylo-oligosaccharides (XOS), fructo-oligosaccharides (FOS), galacto-oligosaccharides (GOS), and combinations thereof; and (b) administering to the companion animal an effective amount of the said food composition.

In certain embodiments, the present disclosure provides a method for improving and/or increasing and/or eliciting the immune response of a companion animal to an antigen, the method including: (a) providing a food composition including at least, in % by weight of the food composition on dry matter basis, i) nucleotides in an amount of at least about 0.05% and ii) oligosaccharides in an amount of at least about 0.1%, wherein said oligosaccharides are selected from xylo-oligosaccharides (XOS), fructo-oligosaccharides (FOS), galacto-oligosaccharides (GOS), and combinations thereof, and (b) administering to the companion animal an effective amount of the said food composition.

In some embodiments, the said immune response can be the immune response of a companion animal to an antigen from a pathogen or a vaccine directed against said pathogen.

In some embodiments, the said immune response can be the immune response of a companion animal to an antigen from one or more pathogens and/or microorganisms and/or a vaccine directed against said pathogen(s) and/or microorganism(s), which can be selected from, without limitation: feline panleukopenia virus, herpesvirus, calicivirus and rabies virus or combination thereof. In some embodiments, the said immune response can be the immune response of a companion animal to an antigen from one or more bacteria selected from the genus Chlamydia, such as without limitation Chlamydophila felis.

In some embodiments, the present disclosure provides a method for preventing or treating immune deficiency in a companion animal, the method including: (a) providing a food composition including at least, in % by weight of the food composition on dry matter basis, i) nucleotides in an amount of at least about 0.05% and ii) oligosaccharides in an amount of at least about 0.1%, wherein said oligosaccharides are selected from xylo-oligosaccharides (XOS), fructo-oligosaccharides (FOS), galacto-oligosaccharides (GOS), and combinations thereof, and (b) administering to the companion animal an effective amount of the said food composition.

In certain embodiments, the present disclosure provides a method for eliciting an immune response in a companion animal, the method including: (a) providing a food composition including at least, in % by weight of the food composition on dry matter basis, i) nucleotides in an amount of at least about 0.05% and ii) oligosaccharides in an amount of at least about 0.1%, wherein said oligosaccharides are selected from xylo-oligosaccharides (XOS), fructo-oligosaccharides (FOS), galacto-oligosaccharides (GOS), and combinations thereof, and (b) administering to the companion animal an effective amount of the said food composition.

In certain embodiments, the present disclosure provides a method as defined above, such as for preventing or reducing the likelihood of occurrence of an infection and/or an allergic reaction in a companion animal, the method including: (a) providing a food composition including at least, in % by weight of the food composition on dry matter basis, i) nucleotides in an amount of at least about 0.05% and ii) oligosaccharides in an amount of at least about 0.1%, wherein said oligosaccharides are selected from xylo-oligosaccharides (XOS), fructo-oligosaccharides (FOS), galacto-oligosaccharides (GOS), and combinations thereof, and (b) administering to the companion animal an effective amount of the said food composition.

According to another aspect, the present disclosure relates to a kit for manufacturing a food composition, including:
(i) a source of nucleotides;
(ii) a source of oligosaccharides selected from xylo-oligosaccharides (XOS), fructo-oligosaccharides (FOS), galacto-oligosaccharides (GOS), and combinations thereof.

Advantageously, the said kit can be provided in a manner, wherein the source of nucleotides and oligosaccharides can be mixed, and preferably, heated, in order to directly manufacture the food composition. In that regard, the skilled in the Art will adapt the content of the respective sources in nucleotides and oligosaccharides with respect to the estimated final amount required in the food composition.

The kit may be adapted for use as a medicament; and/or for use in a method for improving the immune function of a companion animal according to the present disclosure.

According to various embodiments, the present disclosure relates to a method of manufacturing a food composition, including:
a) mixing
   a source nucleotides;
   a source of oligosaccharides selected from xylo-oligosaccharides (XOS), fructo-oligosaccharides (FOS), galacto-oligosaccharides (GOS), and combinations thereof;
b) heating the mixture, thereby manufacturing the food composition

### Definitions

The terms used in this specification generally have their ordinary meanings in the art, within the context of this subject matter and in the specific context where each term is used. Certain terms are defined below to provide additional guidance in describing the compositions and methods of the disclosed subject matter and how to make and use them.

As used herein, the singular forms “a”, “an”, and “the” include plural referents unless the context clearly dictates otherwise. Thus, for example, reference to “a compound” includes mixtures of compounds.

As used herein, the terms “include”, “including”, “comprises”, “comprising” or any other variation thereof, are intended to cover a non-exclusive inclusion, such that a process, method, article, or apparatus that includes a list of elements does not include only those elements but can include other elements not expressly listed or inherent to such process, method, article, or apparatus.

The term “about” or “approximately” means within an acceptable error range for the particular value as determined by one of ordinary skill in the art, which will depend in part on how the value is measured or determined, i.e., the limitations of the measurement system. For example, “about” can mean within three or more than three standard deviations, per the practice in the art. Alternatively, “about” can mean a range of up to 20%, preferably up to 10%, more preferably up to 5%, and more preferably still up to 1% of a given value. Also, particularly with respect to systems or processes, the term can mean within an order of magnitude, preferably within five-fold, and more preferably within two-fold, of a value.

Moreover, the terms “at least”, and “ranging from”, encompass the hereafter cited value. For example, “at least 40 ppm” has to be understood as also encompassing “40 ppm”. Unless specifically stated otherwise, amounts (in particular weight percentages, amount in parts per million (ppm), or milliequivalents/kg (mEq/kg) fat) are expressed herein by weight of a product or composition reference, for example a preservative food composition according to the disclosure. In the present disclosure, ranges are stated in shorthand, so as to avoid having to set out at length and describe each and every value within the range. Any appropriate value within the range can be selected, where appropriate, as the upper value, lower value, or the terminus of the range. For example, a range from 1 to 10 represents the terminal values of 1 and 10, as well as the intermediate values of 2, 3, 4, 5, 6, 7, 8, 9, and all intermediate ranges encompassed within 1-10, such as 2 to 5, 2 to 8, 7 to 10, etc.

In the description herein, references to “embodiments,” “an embodiments,” “one embodiment,” “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

As used herein, the term “animal” refers to animals including, but not limited, to human animals and non-human animals. Preferably animals refer to non-human animals, in particular companion animals, i.e. pets, i.e. canine, felines, i.e. dogs, cats, and the like. Domestic dogs and cats are non-limiting examples of animals.

As used herein, the term “mammal” or “mammals” includes a human or an animal. In particular, the term “animal” or “animals” can designate a ruminant, poultry, swine, mammal, horse, mouse, rat, rabbit, guinea pig, hamster, cow, cat or dog, preferably a companion animal, i.e. pet, i.e. cat, dog and the likes.

As used herein, the term “adult” means an animal has passed puberty and reaches its biological maturation point.

As used herein, a “pet animal” or “companion animal” generally includes, or even consists of, a pet mammal. Pet mammals encompass dogs, cats, rabbits, hamsters, guinea pigs, rats and mice. Preferred pet animals herein are feline or canine, especially as dogs and cats.

As used herein, the term “feline” encompasses animals, including pet animals, selected in the group including cheetah, puma, jaguar, leopard, lion, lynx, liger, tiger, panther, bobcat, ocelot, smilodon, caracal, serval and cats. As used herein, cats encompass wild cats and domestic cats, and most preferably domestic cats.

As used herein, the term “canine” encompasses animals, including pet animals selected in the group including recognized dog breeds (some of which are further subdivided), which can include afghan hound, airedale, akita, Alaskan malamute, basset hound, beagle, Belgian shepherd, bloodhound, border collie, border terrier, borzoi, boxer, bulldog, bull terrier, cairn terrier, chihuahua, chow, cocker spaniel, collie, corgi, dachshund, dalmatian, doberman, English setter, fox terrier, German shepherd, golden retriever, great dane, greyhound, griffon bruxellois, Irish setter, Irish wolfhound, King Charles spaniel, Labrador retriever, lhasa apso, mastiff, newfoundland, old English sheepdog, papillion, pekingese, pointer, pomeranian, poodle, pug, rottweiler, St. Bernard, saluki, samoyed, schnauzer, Scottish terrier, Shetland sheepdog, shih tzu, Siberian husky, Skye terrier, springer spaniel, West Highland terrier, whippet, Yorkshire terrier, etc.

The term “ppm” or “parts per million” is used herein according to its conventional meaning. More precisely, it refers herein to a weight amount relative to the total weight of the preservative food composition, or of the animal food product including the preservative food composition (mg/kg) (unless otherwise indicated).

As used herein, the term “medicament” refers to any compound or composition that provides a benefit or therapeutic effect to the subject. This benefit or therapeutic effect can be achieved upon initial application and/or over time with continued use. The term “medicament” is acceptable for use in human or non-human subjects, such as for animal use.

As used herein, the term “preventing” can also encompass the reduction of a likelihood of occurrence, or of re-occurrence of a condition.

As used herein, the term “immune response” refers to the homeostatic mechanism that has the ability to detect and recognize foreign molecules (such as an antigen). The initial response to foreign molecule is termed “innate immunity” and is characterized by the rapid migration of natural killer cells, macrophages, neutrophils, and other leukocytes to the foreign pathogen site. These cells can either phagocytose, digest, lyse or secrete cytokines that lyse pathogens in a short period of time. The innate immune response is not antigen-specific but is generally regarded as the first line of defense against foreign pathogens until an “adaptive immune response” occurs. Both T-cells and B-cells participate in the adaptive immune response. Various mechanisms are involved in the formation of adaptive immune responses. Consideration of all possible adaptive immune response formation mechanisms is beyond the scope of this section; however, some well-characterized mechanisms are antigen B cell recognition, followed by antigen-specific activation to secrete antibodies and T cell activation by binding to antigen presenting cells.

As used herein, the term “eliciting an immune response” shall be understood to refer to the ability of a subject to raise a specific antibody response and/or a specific T-cell response to an antigen. Preferably, the immune response is an antibody response, and especially an antibody response to a vaccine.

As used herein, the term “increasing an immune response” means enhancing the immune response and/or extending the duration of the immune response. Specifically, within the meaning of the present disclosure the term “increasing an immune response” refers to a property or process that increases the magnitude and/or effectiveness of an immunoreactivity for a given antigen. The administration of the antigen can be intentional, e.g., administration of a live vaccine strain.

As used herein, the term “infection” has the meaning generally used and understood by persons skilled in the art and includes the invasion and multiplication of a microorganism, i.e. bacterium, virus, fungi or parasite (such as an antigen), in or on a subject with or without a manifestation of a disease. An infection can occur at one or more sites in or on a subject. An infection can be unintentional, e.g., unintended ingestion, inhalation, contamination of wounds, or intentional, e.g., administration of a live vaccine strain. In particular, this term “infection” can encompass viral infections, parasitic infections (such as those linked to a fungus), and bacterial infections.

Specific examples of viral infections can include, in a non-exhaustive manner, those selected from: rabies virus; cytomegalovirus (CMV) pneumonia, Epstein-Barr virus, varicella-zoster virus, HSV-1 and -2 mucositis, HSV-6 encephalitis, BK-virus hemorrhagic cystitis, viral influenza, respiratory multinuclear virus (RSV), hepatitis A, B, or C.

Examples of fungal infections include, but are not limited to, aspergillosis; cough throat (caused by Candida albicans); cryptococcosis (caused by Cryptococcus); and histoplasmosis. Thus, examples of infectious fungi include Cryptococcus neoformans, Histoplasma capsulatum, Coccidioides immitis, Blastomyces dermatitidis.

Examples of infectious bacteria can include: the genus Chlamydia as a whole (e.g. Chlamydia felis), Helicobacter pylori, Borelia burgdorferi, Legionella pneumophilia, Mycobacteria sps (human tuberculosis) M. tuberculosis, M. avium, Mycobacterium intracellulare, M. kansaii, M. gordonae)), Staphylococcus aureus, Neisseria gonorrhoeae, Neisseria meningitidis, Listeria monocytogenes, Streptococcus pyogenes (Group A streptococcus), Streptococcus⋅Streptococcus agalactiae (Group B Streptococcus), Viridans streptococci (Streptococcus (viridans group)), Streptococcus faecalis, Streptococcus bovis, Streptococcus (anaerobic sps.), Streptococcus pneumoniae, Pathogenic Campylobacter species (Campylobacter species) sp.), Enterococcus sp., Haemophilus influenzae, Bacillus anthracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasteurella multocida, Bacteroides sp. Bacteroides sp. (Fusobacterium nucleatum), Streptobacillus monilformis, Treponema pallidium, Treponema pertenue, Leptospira, and Actinomyces israeli. Other infectious organisms (such as protozoa) include: Plasmodium falciparum, and Toxoplasma gondii.

As used herein, the term “effective amount” refers to an amount of an ingredient which, when included in a composition, is sufficient to achieve an intended compositional or physiological effect. Thus, a “therapeutically effective amount” refers to a non-toxic, but sufficient amount of an active agent, to achieve therapeutic results in treating or preventing a condition for which the active agent is known to be effective. It is understood that various biological factors can affect the ability of a substance to perform its intended task. Therefore, an “effective amount” or a “therapeutically effective amount” can be dependent in some instances on such biological factors. Further, while the achievement of therapeutic effects can be measured by a physician or other qualified medical personnel using evaluations known in the art, it is recognized that individual variation and response to treatments can make the achievement of therapeutic effects a subjective decision. The determination of an effective amount is well within the ordinary skill in the art of pharmaceutical and nutritional sciences as well as medicine and refers to the amount of a conjugate (e.g. carnosic acid, hydroxytyrosol, tannin, ellagic acid, gallic acid) or combination necessary or sufficient to realize the desired biological effect.

As used herein, “administration”, and “administering” refer to the manner in which an active agent, or composition containing such, is presented to a subject. Administration can be accomplished by various routes well-known in the art such as oral and non-oral methods.

As used herein, “oral administration” refers to a route of administration that can be achieved by swallowing, chewing, or sucking of an oral dosage form including the food composition or animal food product. Examples of oral dosage forms include tablets capsules, caplets, powders, granulates, beverages, jelly, kibbles, or other animal food products as mentioned in the present disclosure.

As used herein, a “food composition” refers to any molecules or substances, or combinations of blends thereof, that can be added to food products, including beverages, to prevent undesirable chemical changes.

As used herein, the term “animal food product” or “food product” or “product” refers to a composition or product intended for ingestion by an animal or a pet. Animal food products can include, without limitation, any composition or product which is suitable for daily feed as well as treats, nutritionally balanced or not, and nutritionally complete or not.

Food compositions and animal food products disclosed herein can be dry or wet food. In particular, animal food products food compositions can be dry animal food products or dry food compositions.

As used herein, the term “nutritionally complete” refers to animal food products that contain all known required nutrients for the intended recipient of the animal food product, in all appropriate amounts and proportions based, for example, on recommendations of recognized and competent authorities in the field of animal nutrition. Such foods are therefore capable of serving as a source of dietary intake to maintain life, without the addition of supplemental nutritional sources.

As used herein the term “nutritionally balanced” refers to an animal food product which, through a single or reference serving of the said food, provides a nutritionally desirable level of fat, protein or amino acid source, and dietary fiber.

The term “nutritionally balanced”, as used herein, can thus refer to animal food products that can be nutritionally complete. Alternatively, “nutritionally balanced”, as used herein, can also refer to animal food products that are not nutritionally complete.

As used herein, the term “functional food” refers to a food product which provides nutritional components that are important for health maintenance. These food compositions contain compounds that are biologically active or bioavailable, such as probiotics, amino acids, multivitamins, and antioxidants, and often are found to be useful for the treatment of disease and disorders or the maintenance of normal health states.

As used herein, the terms “dry animal food product” or “dry food composition” generally refer to a food product or composition having a moisture content of less than 12% by weight, relative to the total weight of the food product or composition, and commonly even less than 7% by weight, relative to the total weight of the food product or composition. Dry animal food products can be formed by an extrusion process. In some embodiments, a dry animal food product can be formed from a core and a coating to form a dry animal food product that is coated, also called a coated dry animal food product. It should be understood that when the term “dry animal food product” is used, it can refer to an uncoated dry animal food product or a coated dry animal food product. A dry animal food composition can be a kibble.

As used herein, the term “kibble” includes a particulate pellet like component of animal feeds, such as dog and cat feeds, typically having a moisture, or water, content of less than 12% by weight, relative to the total weight of the kibble. Kibbles can range in texture from hard to soft. Kibbles can range in internal structure from expanded to dense.

As used herein, the term “core”, or “core matrix”, means the particulate pellet of a dry animal food product, i.e. a kibble, and is typically formed from a core matrix of ingredients. The particulate pellet can be coated to form a coating on a core, which can be a coated dry animal food product. The core can be without a coating or can be with a partial coating. In an embodiment without a coating, the particulate pellet can include the entire dry animal food product. Cores can include farinaceous material, proteinaceous material, and mixtures and combinations thereof. In one embodiment, the core can include a core matrix of protein, carbohydrate, and fat.

As used herein, the term “coating” means a partial or complete covering, typically on a core, that covers at least a portion of a surface, for example a surface of a core. In one example, a core can be partially covered with a coating such that only part of the core is covered, and part of the core is not covered and is thus exposed. In another example, the core can be completely covered with a coating such that the entire core is covered and thus not exposed. Therefore, a coating can cover from a negligible amount up to the entire surface. In an embodiment, a food composition of the disclosure can be suitable for the preparation of a dry animal food product by coating.

As used herein, the terms “wet animal food product” or “wet food composition” generally refer to a food product or composition having a moisture content of higher than 12% by weight, relative to the total weight of the food product or composition, and commonly even higher than 30% by weight, relative to the total weight of the food product or composition.

As used herein, the term “semi-moist food” or “semi-moist food composition” or “semi-moist food product” particularly refers to a food composition with an intermediate moisture content of about 12% to about 30% in weight, relative to the total weight of the food composition. Hence, such semi-moist food composition is generally the final product of a process allowing a moisture content value that is intermediate between a dry food and a wet food. In some embodiments, the said process can include a step of adding a humectant agent. In some embodiments, the said process includes an extrusion step and a subsequent treatment step with Super-Heated Steam (SHS). In some embodiments, the semi-moist food according to the present disclosure containing more than 12% and at most 30% moisture by weight, relative to the total weight of the food composition. Illustratively, a semi-moist food composition has 11% to 25% moisture by weight, relative to the total weight of the food composition.

As used herein, an “extrudate” refers to any product, such as an animal food product, which has been processed by, such as by being sent through, an extruder or pelleting process. An extrudate can be dry or wet. Preferably, an extrudate is a dry extruded product. In a particular embodiment, an extruded product is a dry animal food product, in particular a kibble. In one embodiment of extrusion, kibbles are formed by an extrusion process wherein raw materials, including starch, can be extruded under heat and pressure to gelatinize the starch and to form the pelletized kibble form, which can be a core. Any type of extruder can be used, non-limiting examples of which include single screw extruders and twin-screw extruders.

As used herein, the terms “palatability” or “palatable” refer to being desirable to the palate or taste. Further, the terms “palatability” or “palatable” as used herein refer to the extent to which a pet food product appeals to the palate or taste of an animal. This is suitable measured by feeding tests, e.g., difference tests or ranking tests. In certain embodiments, “palatability” can mean a relative preference for one food product over another. For example, when an animal shows a preference for one of two or more food products, the preferred food product is more “palatable”, and has “enhanced palatability” or “increased palatability”. In certain embodiments, the relative palatability of one food product compared to one or more other food products can be determined, for example, in side-by-side, free-choice comparisons, e.g., by relative consumption of the food products, or other appropriate measures of preference indicative of palatability, i.e. “the two-bowl test”.

As used herein, the term “protein source” can encompass “animal protein sources”, “plant protein sources”, or any other amino acid source, or combinations thereof. According to some embodiments, the protein source can include or can consist of hydrolyzed proteins; e.g. partially or totally hydrolyzed proteins. Methods to hydrolyze, partially or totally proteins are well-known. A hydrolysate can be produced by any known chemical or enzymatic method, such as, in non-limiting example, methods disclosed in U.S. Pat. Nos. 5,589,357, 4,879,131, 5,039,532 or EP 1 236 405 Bl. According to some embodiments, the protein source is not hydrolyzed.

As used herein, the term “amino acid” can refer to any naturally-occurring or non-naturally-occuring amino acid. In a non-exhaustive manner, the amino acid can be selected from the group consisting of: Alanine, Arginine, Asparagine, Aspartate, Cysteine, Glutamate, Glutamine, Glycine, Histidine, Isoleucine, Leucine, Lysine, Methionine, Phenylalanine, Proline, Serine, Taurine, Threonine, Tryptophan, Tyrosine, and Valine or a combination thereof.

As used herein, the term “amino acid source” means a material containing amino acids. Said amino acid source can include or be derived from, but is not limited to, plant proteins, animal proteins, proteins from single cell organisms and free amino acids.

As used herein, the term “animal protein” refers to animal-based sources of protein. Such animal protein includes, for example without limitation, meat (for example, pork, beef, or veal), poultry (for example, chicken), fish, organs (for example, liver, spleen, or heart), viscera (for example, viscera of chicken or pork), and combinations thereof. As animal proteins, one can select, in a non-exhaustive manner, animal proteins from poultry, beef, chicken, chicken meal, lamb, lamb meal, dried egg, fish, fish meal, meat and bone meal, meat byproducts, meat meal, turkey, blood plasma or bone marrow.

As plant or vegetal proteins, one can select, in a non-exhaustive manner, vegetal proteins from soybean, chickpea, pea, corn gluten, rice, insects, lentils, or barley.

As used herein, the term “antioxidant” refers to any molecule, composition or products which delays or prevents the oxidation of an animal food product, and in particular of an oxidizable fat. Antioxidant food compositions or products of the present disclosure prevent or delay the oxidation process. Further, antioxidants preserve fresh attributes and nutritional quality of the corresponding food composition or product. Antioxidants of the present disclosure can include or can consist of synthetic or natural antioxidants. Advantageously, food compositions or products can include lesser amounts of synthetic antioxidants. According to some embodiments, such antioxidant food compositions can include minimal, or undectable, amounts of synthetic antioxidants. For example, such antioxidant food compositions can include synthetic antioxidants in amounts less than about 1% in weight of the total weight of the corresponding composition. Advantageously, the antioxidants which can be present in such food compositions, or products, consist exclusively of non-synthetic (i.e. natural) antioxidants.

As used herein, the term “synthetic antioxidant” refers to chemically synthesized, non-naturally occurring, compounds which can be added to food as preservatives to help prevent lipid oxidation. In a non-exhaustive manner, this term thus encompasses the following compounds: Butylated hydroxytoluene (BHT), butylated hydroxyanisole (BHA), TBHQ (tert-butylhydroxyquinone), propyl gallate (PG), dodecyl gallate (DG), octylgallate (OG) and chelating agent, such as ethylenediaminetetraacetic acid (EDTA).

As used herein, the term “natural antioxidant” refers to naturally-occurring compounds with antioxidant properties.

The “antioxidant” properties of a given product or composition of the present disclosure can be assessed by determining its ability to delay or prevent the oxidation of a molecule such as a lipid, lipoprotein, protein or DNA, over a given length of time.

As used herein, the term “fat” refers to the total amount of digestible, partially digestible and nondigestible fats or oils that are present in the embodiments of the present disclosure; especially the food products or compositions for which fat oxidation should be prevented or delayed. As used herein, the terms “lipid”, “fat” and “oil” are synonymous.

The constituents of oils and fats are known in chemistry to possess a tendency to absorb and react with oxygen. The development of rancidity results primarily from the products formed during oxidation. The dissolved or absorbed oxygen usually reacts first to form peroxides. The development of peroxides is accelerated by moisture, heat, light or catalysts. Aldehydes, ketones and acids of lower molecular weight are formed in the further decomposition and these materials impart an undesirable odor and taste to the oil or fat.

For quality assessment, methods known by the skilled person, such as peroxide value determination (PV), hexanal value determination, ferric thiocyanate method (FTC), thiobarbituric acid method (TBA), anisidine index determination, conjugated dienes determination, or any method for determining the stability such as oxygen bomb or rancimat. According to a preferred embodiment, the determination of major primary products (i.e. hydroperoxides) resulting from lipid oxidation, as well as secondary compounds (including alkanes, alkenes, aldehydes, ketones, alcohols, esters, acids and hydrocarbons) can thus be used to assess antioxidant properties. In a non-exhaustive manner, those antioxidant properties can thus be assessed by determining a “peroxide value” (PV), or an “hexanal value”.

As used herein “peroxide value” (PV) refers to the marker for fatty acids primary oxidation degradation compounds. Otherwise said, PV is used for the quantification of primary fat-oxidation products. Peroxide values of fresh food products are less than about 10 milliequivalents/kg (mEq/kg) whereas when the peroxide value is between about 20 and about 40 mEq/kg, the food product is considered rancid. According to a preferred embodiment, these values must be determined at end of shelf-life. According to an embodiment, a value up to 10 mEq/kg will be considered as rancid. Methods to analyze the PV of an animal food product are well known by the skilled person. Illustratively, the skilled person can use the NF EN ISO 3960 (Version of April 2017).

As used herein “hexanal value” refers to the marker for fatty acids second oxidation degradation compounds. Hexanal values of fresh food products are less than about 15 ppm whereas when the hexanal value is between about 15 and about 40 ppm, the food product is considered rancid. According to a preferred embodiment, these values have also to be determined at end of shelf-life. According to an embodiment, a value up to 15 ppm will be considered as rancid. Methods to analyze the hexanal level of an animal food product are well known by the skilled person. Illustratively, the skilled person can use the AOCS method Cg 4-94 (AOCS. 1997).

As used herein, and unless specified otherwise, the term “nucleotides” can refer in particular to 5′ nucleotides, which are nucleotides with a phosphoric acid group in the 5′-position of ribose. A “nucleotide” is preferably understood to be a subunit of deoxyribonucleic acid (“DNA”) or ribonucleic acid (“RNA”). Such 5′nucleotides can, for instance, be obtained through treatment of a starting material with 5′ nucleotidases and/or phosphatases. In a non-exhaustive manner, a 5′nucleotide according to the present disclosure can thus be selected from the group consisting of: adenosine monophosphate (AMP), guanosine monophosphate (GMP), inosine monophosphate (IMP), uridine monophosphate (UMP), cytidine monophosphate (CMP), thymidine monophosphate (TMP), xanthosine monophosphate (XMP) or a mixture of two or more thereof. For example, the nucleotide can be AMP, GMP, or IMP or a mixture thereof, or the nucleotide can be GMP alone, or IMP alone, or a mixture thereof.

As used herein, and unless specified otherwise, the term “nucleotide source” refers to any source, in particular natural source, natural, processed or provided as raw material which contains nucleotides as defined above. For example, a nucleotide source suitable for the present disclosure can be hydrolyzed; e.g. partly or completely hydrolyzed. For example, a nucleotide source suitable for the present disclosure can include or can consist of a biological extract such as a bacterial extract or a yeast extract source of nucleotides. In a non-exhaustive manner, the yeast can be selected from Kluyveromyces or Saccharomyces cerevisiae. Examples of such nucleotide sources can thus include or can consist of petMOD™ and/or petMOD™S feed material sources, which are commercialized by PROSOL S.P.A. Quality Control of Yeast Extract Nucleotides can be achieved by any method known in the Art, such as ion-pair high-performance liquid chromatography.

Also, as used herein, the term “polynucleotide” refers to a polymeric form of nucleotides of any length, either ribonucleotides or deoxynucleotides, and includes both double- and single-stranded DNA and RNA. Accordingly, the terms “nucleotide” and “5′nucleotide” are used throughout the specification distinctly from polynucleotides.

As used herein, and unless specified otherwise, the term “oligosaccharide” refers to a compound containing two or more monosaccharide units linked by glycosidic bonds. Accordingly, it can refer to all types of oligosaccharides, such as those found from natural and synthetic sources, including those susceptible to be obtained through microorganisms.

In the context of the present disclosure, and especially, the food compositions and products according to the present disclosure, the oligosaccharides which can be considered include those selected from: a gluco-oligosaccharide, a galacto-oligosaccharide, a fructo-oligosaccharide, a manno-oligosaccharide, an arabino-oligosaccharide, a xylo-oligosaccharide, or any combinations thereof.

Examples of oligosaccharides can further include functionalized oligosaccharides. Functionalized oligosaccharide compositions can be produced by, for example, combining one or more sugars with one or more functionalizing compounds in the presence of a catalyst.

More particularly, especially in the context of a combination with nucleotides, the oligosaccharides which are specifically considered are those selected from: a galacto-oligosaccharide (GOS), a fructo-oligosaccharide (FOS), a xylo-oligosaccharide (XOS), or any combinations thereof.

The oligosaccharides according to the present disclosures can, in particular, be characterized based on the type of oligosaccharides present and/or the degree of polymerization (DP). The oligosaccharide content of reaction products resulting from polymerization can be determined, e.g., by a combination of high-performance liquid chromatography (HPLC) and spectrophotometric methods. For example, the average degree of polymerization (DP) for the oligosaccharides can be determined as the number average of species containing one, two, three, four, five, six, seven, eight, nine, ten to fifteen, and greater than fifteen, anhydrosugar monomer units

As used herein, the term “long oligosaccharide” refers to an oligosaccharide composition with an average degree of polymerization (DP) of about 8, about 9, about 10, about 11, about 12, about 13, about 14, about 15, about 16, about 17, about 18, about 19, or about 20.

As used herein, the term “short oligosaccharide” refers to an oligosaccharide with an average degree of polymerization (DP) of about 2, about 3, about 4, about 5, about 6, or about 7. In particular, the short oligosaccharide can refer to a short-chain fructo-oligosaccharide (scFOS). Examples of scFOS can thus include 1-kestose, nystose, 1F-β-fructofuranosyl nystose, or combinations thereof. For example, scFOS according to the present disclosure can include short-chain fructooligosaccharides (scFOS) obtained from sucrose; consisting of a combination of 1-kestose (GF2) (about 37%), nystose (GF3) (about 53%), and 1F-β-fructofuranosyl nystose (GF4) (about 10%). GF2, GF3, and GF4 are formed by a sucrose (glucose-fructose) molecule linked to respectively one, two or three fructose units added by β2-1 glycosidic linkages to the fructose unit of sucrose.

As used herein, and unless specified otherwise, the term “arginine” can refer to any arginine or arginine source, in particular any L-arginine or L-arginine source, which can be used i.e., synthetic or natural. Food ingredients which can include L-arginine in significant amounts can include for example nut meats, nut pastes, and/or nut flours, seeds, seed pastes, and/or seed flours, or gelatin. For example, L-arginine can be derived from any available arginine source, e.g., Arachis hypogaea (peanuts), Juglans regia (walnuts), Prunus amygdalus (almonds), Corylus avellana (hazelnuts), Glycine max (soybean), Carya illinoensis (pecans), Amacardium occidentale (cashews), and Macadamia integrifolia, M. tetraphylla (macadamia nuts) and the like.

### EXAMPLES

The presently disclosed subject matter will be better understood by reference to the following Examples, which are provided as exemplary of the disclosure, and not by way of limitation. The materials and methods used in the examples are summarized below.

### Material & Methods

Vaccination protocol for kittens (European cats). Pregnant queens have been fed ad libitum with the control diet from the detection of pregnancy until their kittens were weaned. At the beginning of weaning, at week 4, kittens were divided in two groups. One is fed with the control diet and the other one with the supplemented diet. From 4 to 8 weeks of age, the percentage of solid food will increase progressively to finally reach 100% dry food. For this study, 50 kittens have been allotted in two groups of 25. Since we could not separate the kittens from their mothers, the allotment has been performed according to the sex and the weight in order to have two homogeneous groups:

TABLE 1
Tested animal populations, and control groups.

| | Number of | Average female | Number of | Average |
|---|---|---|---|---|
| | Females | weight | Males | male weight |
| Group 1 | 10 | 0.408 g | 15 | 0.455 g |
| Group 2 | 10 | 0.397 g | 15 | 0.461 g |

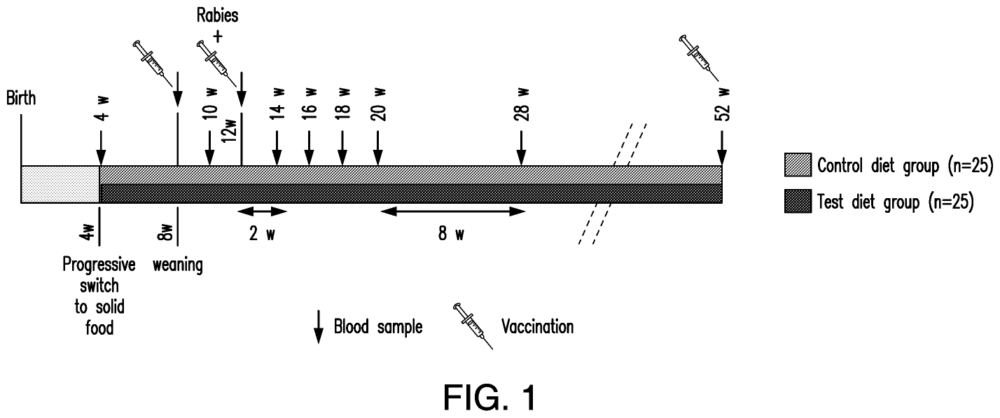

The administration protocol was administered as in FIG. 1. Tested nutritional solutions are administered from the beginning of weaning until the kittens are one year old. The control diet group and the supplemented diet groups (n=25 each) are progressively switched to solid food. Kittens are vaccinated at weeks 8, 12 and 52 weeks with a veterinary vaccine against feline viral rhinotracheitis, feline calicivirosis, feline chlamydiosis and feline panleucopenia (PUREVAX RCPCh), following classical veterinarian protocol. At week 12, they are also vaccinated with a veterinary vaccine including inactivated Rabies virus (Rabisin®). Blood samples, are collected at 4 weeks, 8 weeks, 10 weeks, 14 weeks, 16 weeks, 18 weeks, 20 weeks, 28 weeks, 36 weeks, 44 weeks and 52 weeks.

As mentioned, the tested nutritional solutions were given from the beginning of weaning until the kittens are one year old. Kittens were vaccinated at weeks 8, 12 and 52 with a PUREVAX RCPCh vaccine, following the classical veterinarian protocol. At week 12, they were also vaccinated with Rabisin®. During the whole year, the weight of the animals was monitored weekly to establish a growth curve and be able to compare the growth between the different groups. The volume of blood (whole blood and serum) was collected and measured according to the age and weight of the kittens. The following parameters were determined: whole blood cells, antiviral & antibacterial antibodies, a set of cytokines measured by whole blood assay, including sFas, Flt-3 ligand, GM-CSF, IFN-γ, IL-1β, IL-2, IL-4, IL-6, IL-8, IL-12 (p40), IL-13, IL-18, MCP-1, PDGF-BB, RANTES, SCF, TNF-α. IL-10. Whole blood cell parameters were statistically determined as defined hereafter. Statistically significant compared to control: *=p-value <0,05; **=p-value <0,01; *=p-value <0,001; ****=p-value <0,0001

### Measurement of Whole Blood Cells.

This measurement was performed at each time points in order to follow all blood parameters (White Blood Cells, Red Blood Cells, % of Neutrophils, % of Lymphocytes, % of Monocytes, % of Eosinophils, % of Large Unstained Cells, % of Basophils, % of Reticulocytes, Platelet Count, Haemoglobin and Hematocrit) of each kitten during the whole study. The aim of following those parameters was to make sure that the kittens were healthy during the whole study.

### Measurement of Antiviral/Antibacterial Antibodies in Plasma

Responses to Panleucopenia, Chlamydia, herpesvirus, calicivirus and rabies vaccines were evaluated by measuring specific immunoglobulin levels by ELISA in blood samples collected before the primo-vaccination (week 8), then every two weeks until week 20 and finally every 8 weeks until the end of the protocol.

### Measurement of Cytokine Expression.

The production of cytokines was monitored on Whole Blood Assay as follows: the heparinized whole blood was 1:1 diluted with R-10 culture cell medium, then 100 μL of each diluted blood sample were pipetted into 96-well flat-bottom plates. The blood samples were cultivated in duplicates in 3 different conditions: without any stimulation, stimulated with PHA (Phytohaemagglutinin) at 5 μg/mL as a non-specific stimulus for T lymphocytes, and rabies virus antigen at 5 μg/mL as a specific stimulus (Recombinant Rabies virus Glycoprotein (aa 20-458)[His] (DAGF-021) Creative Diagnostics). The plates were incubated for 2 days at 37° C. in a humidified incubator under 5% C02 atmosphere. After the incubation period, the plates were centrifuged, and the supernatants harvested for Luminex and IL-10 assays. The plates were read by INc's MAGPIX® instrument using the MAGPIX® software (xPONENT MAGPIX). The quantification was performed using the MILLIPLEX® Analyst 5.1 software.

The following cytokines were analyzed by Luminex: sFas, Flt-3 ligand, GM-CSF, IFN-γ, IL-1β, IL-2, IL-4, IL-6, IL-8, IL-12 (p40), IL-13, IL-18, MCP-1, PDGF-BB, RANTES, SCF, TNF-α. IL-10 levels were assayed by ELISA.

### Example 1. Food Composition Formulation

One possible combination of a nucleotide source and of sources of oligosaccharides according to the present disclosure is provided hereafter. The composition corresponds the food supplement administered to kittens during the vaccination protocol.

TABLE 2
Examples of food formulations

| | Amount in | Weight in % |
|---|---|---|
| | mg per kg of | of the total |
| | dry matter of the | weight of the |
| Active Ingredient | composition | composition |
| scFOS | 4875 mg | 0.5% |
| XOS | 3000 mg | 0.3% |
| Yeast extract enriched in | — | 0.33% |
| nucleotides | | |
| Purines (AMP + GMP + IMP) | 514 mg | 0.05% |
| Pyrimidines (CMP + UMP) | 626 mg | 0.06% |
| Total amount of nucleotides | 1130 mg | 0.11% |
| Betacarotene | 7 mg | 0.0007% |
| Vitamin E | 280 mg | 0.03% |

| | |
|---|---|
| Arginine | Level reached by the other ingredients |

The yeast-extract enriched in nucleotides was selected from petMOD™ and petMOD™S feed material sources, which are commercialized by PROSOL S.P.A.

The xylo-oligosaccharide (XOS) source was a XOS powder composition including XOS (35% to 40% of dry weight of the source) in combination with maize maltrodextrin (30 to 35%), maize fiber including xylose and xylan (25 to 35%).

The short-chain fructo-oligosaccharide (scFOS) source was a PROFEED® composition including short-chain fructooligosaccharides (sc-FOS) obtained from sucrose. They consisted of a combination of 1-kestose (GF2) (about 37%), nystose (GF3) (about 53%), and 1F-β-fructofuranosyl nystose (GF4) (about 10%). GF2, GF3, and GF4 are formed by a sucrose (glucose-fructose) molecule linked to respectively one, two or three fructose units added by β2-1 glycosidic linkages to the fructose unit of sucrose.

### Example 2. Assessment of the Effect of the Combination on Vaccine Response in Kittens on Humoral Immune Response & Antibody Production

As defined in FIG. 1, the administered vaccines included:
a veterinary vaccine against feline viral rhinotracheitis, feline calicivirosis, feline chlamydiosis and feline panleucopenia (PUREVAX RCPCh);
a veterinary vaccine including inactivated Rabies virus (Rabisin®).

Accordingly, the administration included, in total, two different vaccine compositions directed against five distinct pathogens, consisting of one type of pathogenic bacteria (Chlamydia) and four distinct pathogenic viruses.

The profiles of the response to vaccines were normal for all the groups and all the valences tested. It has been shown an improvement of vaccine response for the Chlamydia vaccine for the group of kittens fed with the diet containing the combination of active ingredients including the nucleotide source (enriched yeast-extract) and the selection of oligosaccharides including scFOS and XOS (also reported herein as “cocktail”).

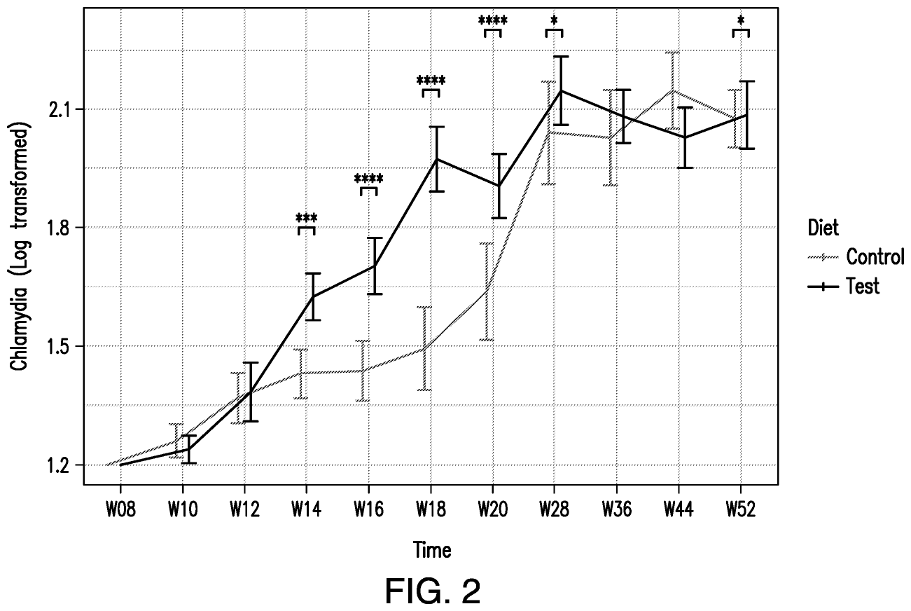

The FIG. 2 illustrates a statistically very significant improvement in the tested group (p-value <0.001) at week 14 in terms of antibody production and an outstanding improvement (p-value <0.0001) at week 18. This provides evidence that the cocktail particularly improves the humoral immune response for vaccines directed against bacteria, such as Chlamydia.

There was no impact of the tested cocktail/combination on the body weight of the growing kittens between the tested and control groups.

### Example 3. Assessment of the Biological Effect of the Combination on Other Physiological Parameters & Whole Blood Assays

The expression of a set of cytokines was assessed ex vivo on blood samples performed at weeks 8, 12, 16 and 20 and cultured in 3 conditions:
not stimulated
stimulated with PHA
stimulated with rabies antigen (RA)

Twenty distinct cytokines were tested with the Luminex® assay. Seven of them were not detectable: sFas, IFN-γ, IL-4, IL-13, PDGF-BB, SCF and SDF-1. Signals were obtained for 13 others, namely: IL12p40 (natural killer cell stimulatory factor 2), IL-6, RANTES (Regulated on Activation, Normal T cell Expressed and Secreted), IL-8 (neutrophil chemotactic factor), KC (keratinocytes-derived chemokine), IL-2, MCP1 (monocyte chemoattractant protein 1), GMCSF (Granulocyte-macrophage colony-stimulating factor), IL-18 (interferon-gamma inducing factor), TNF-α, FLT3L (FMS-like tyrosine kinase 3 ligand), IL-1β, IL-10.

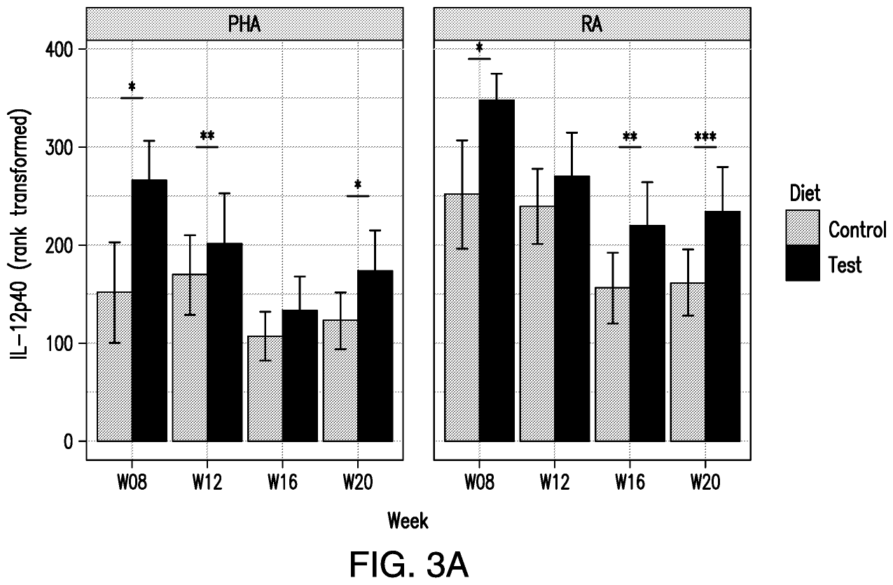

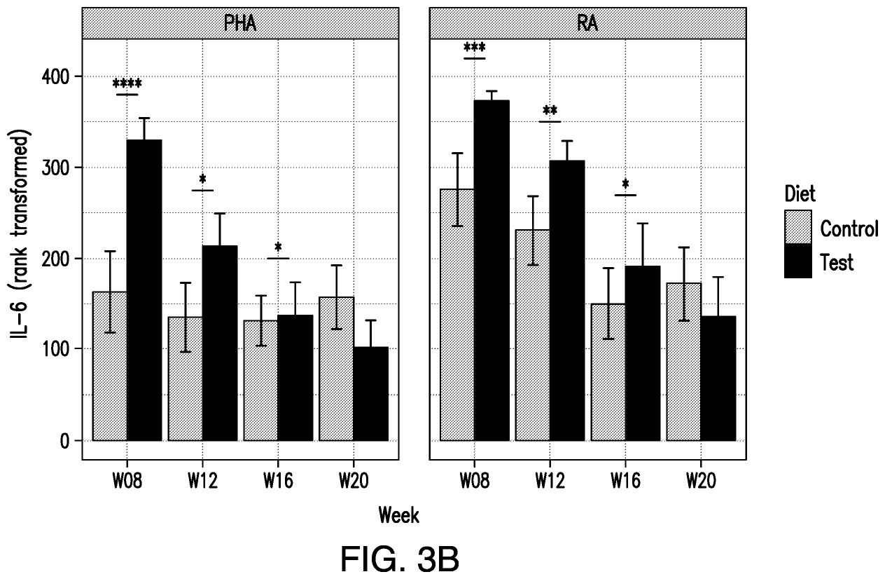

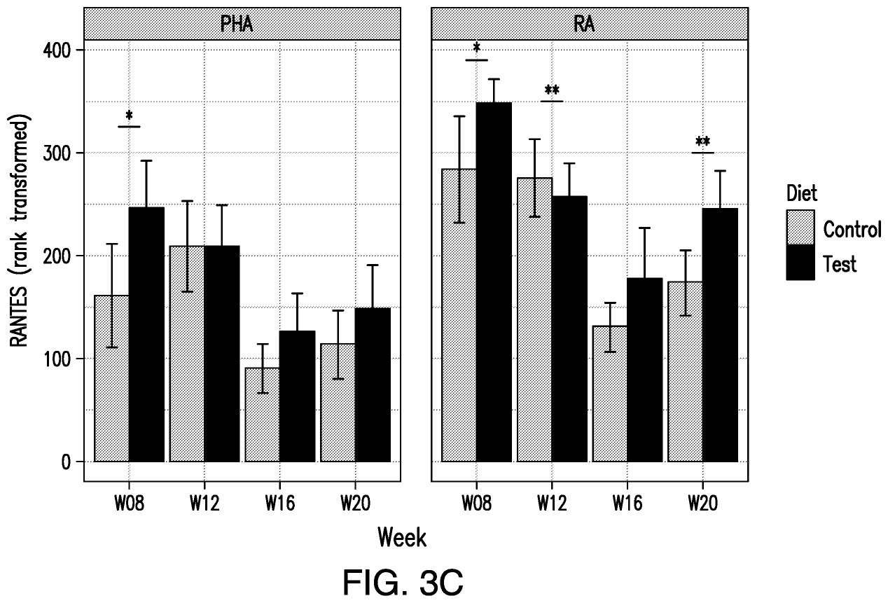

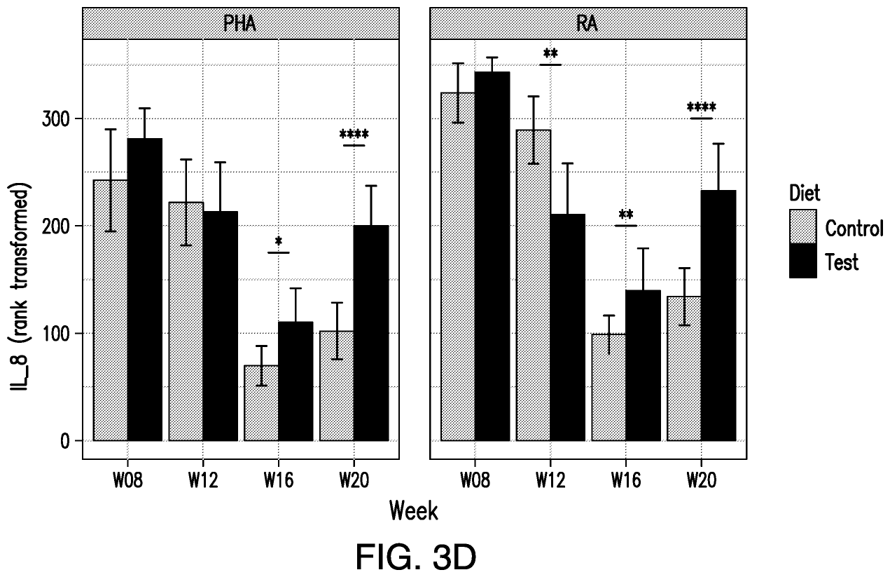

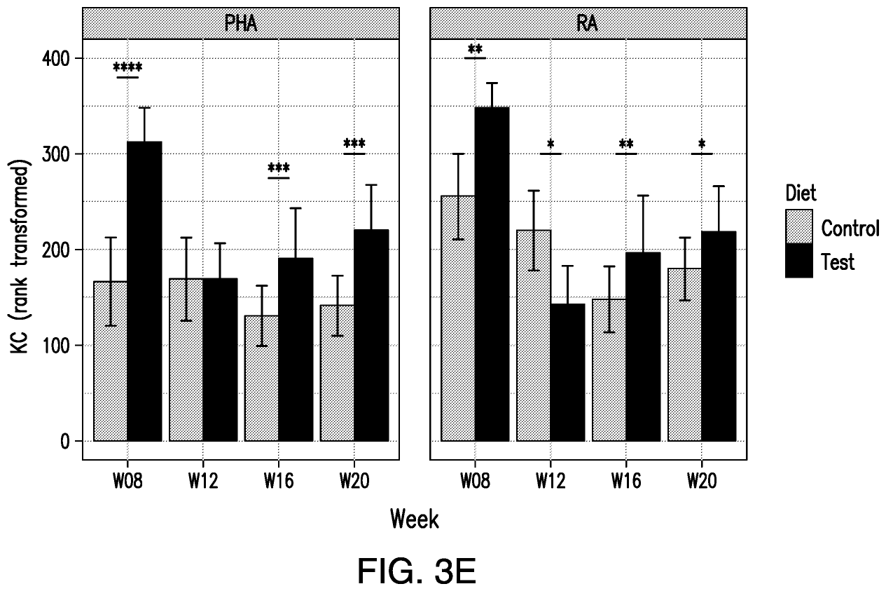

FIG. 3 illustrates the results for both groups (tested vs. control) that are provided in separate panels for the following cytokines: 3A. IL12p40 (natural killer cell stimulatory factor 2). 3B. IL-6. 3C. RANTES (Regulated on Activation, Normal T cell Expressed and Secreted). 3D. IL-8 (neutrophil chemotactic factor). 3E. KC (keratinocytes-derived chemokine). For each graph, the y-axis represents the Luminex signal in ng per million of cells. Grey bars correspond to the control group and black bars to the test group. For each group, four measurements were achieved in duplicates, with from left to right: Week 8, Week 12, Week 16 and Week 20

Among the 13 detectable cytokines, IL12p40 (FIG. 3A), IL-6 (FIG. 3B), RANTES (FIG. 3C), IL-8 (FIG. 3D) and KC (FIG. 3E) exhibited strong responses to the rabies antigen stimulation in the supplemented group compare to the control group.

Interestingly, those 5 cytokines are involved in immune cell proliferation or chemotaxis. This may mean that the immune cells in the supplemented group had, ex vivo, improved capacities compared to the control group.

These results show that after a viral antigenic stimulation the individuals of the supplemented group have a better immune response through the cytokines expressed.

1. A food composition comprising, in % by weight of the food composition on dry matter basis, i) nucleotides in an amount of at least about 0.05% and ii) oligosaccharides in an amount of at least about 0.1%, wherein said oligosaccharides are selected from xylo-oligosaccharides (XOS), fructo-oligosaccharides (FOS), galacto-oligosaccharides (GOS), and combinations thereof.

2. The food composition of claim 1, comprising, in % by weight of the food composition on dry matter basis, i) nucleotides in an amount ranging from about 0.05% to about 0.5%, and ii) oligosaccharides in an amount ranging from about 0.1% to about 2%, wherein said oligosaccharides are selected from xylo-oligosaccharides (XOS), fructo-oligosaccharides (FOS), galacto-oligosaccharides (GOS), and combinations thereof.

3. The food composition of claim 1, comprising, in % by weight of the food composition on dry matter basis:
(i) nucleotides in an amount ranging from about 0.05% to about 0.5%;
(ii) xylo-oligosaccharides (XOS) in an amount ranging from about 0.1% to about 0.7%, and/or
(iii) fructo-oligosaccharides (FOS) in an amount ranging from about 0.3% to about 1%.

4. The food composition of claim 1, wherein the nucleotides are from a source of nucleotides selected from a yeast extract or a fraction thereof.

5. The food composition of claim 1, wherein said nucleotides comprise a combination of pyrimidines and purines.

6. The food composition of claim 1, wherein the oligosaccharides are selected from xylo-oligosaccharides (XOS), fructo-oligosaccharides (FOS), and combinations thereof.

7. The food composition of claim 1, wherein said XOS comprise XOS composed of xylose units linked by β-(1,4) bonds, with a degree of polymerization (DP) ranging from 2 to 10.

8. The food composition of claim 1, further comprising
vitamin E in an amount ranging from about 0.01% to about 0.1%, in % by weight of the food composition on dry matter basis.

9. The food composition of claim 1, wherein said food composition is a dry composition, a wet composition, or a semi-moist composition.

10. The food composition of claim 1, wherein the food composition is a medicament.

11-17. (canceled)

18. The food composition of claim 4, wherein the yeast extract is selected from a Kluyveromyces yeast extract or a Saccharomyces cerevisae yeast extract, or a fraction thereof.

19. The food composition of claim 7, wherein said FOS comprise at least about 50% of short-chain fructo-oligosaccharides (scFOS) in % by weight of the FOS in the food composition on dry matter basis.

20. The food composition of claim 1, further comprising beta-carotene in an amount ranging from about 0.0001% to about 0.001%, in % by weight of the food composition on dry matter basis.

21. The food composition of claim 1, further comprising arginine in an amount ranging from about 0.1% to about 10%, in % by weight of the food composition on dry matter basis.

22. The food composition of claim 9, wherein said food composition is a dry composition.

23. A method for improving the immune function of a companion animal, comprising administering to the companion animal a food composition comprising, in % by weight of the food composition on dry matter basis, i) nucleotides in an amount of at least about 0.05% and ii) oligosaccharides in an amount of at least about 0.1%, wherein said oligosaccharides are selected from xylo-oligosaccharides (XOS), fructo-oligosaccharides (FOS), galacto-oligosaccharides (GOS), and combinations thereof.

24. The method of claim 23, wherein the immune function is selected from: cytokine production and antibody response to a vaccine, the immune response to an antigen, and combinations.

25. A method for preventing or treating immune deficiency in a companion animal, comprising administering to the companion animal a food composition comprising, in % by weight of the food composition on dry matter basis, i) nucleotides in an amount of at least about 0.05% and ii) oligosaccharides in an amount of at least about 0.1%, wherein said oligosaccharides are selected from xylo-oligosaccharides (XOS), fructo-oligosaccharides (FOS), galacto-oligosaccharides (GOS), and combinations thereof.
